(19) United States
(12) Patent Application Publication
(10) Pub. No.: US 2013/0117889 A1
(43) Pub. Date: May 9, 2013

(54) POLYNUCLEOTIDE, POLYPEPTIDE SEQUENCES AND METHODS THEREOF

(71) Applicants: Khareedu Venkateswara Rao, Hyderabad (IN); Vudem Dashavantha Reddy, Hyderabad (IN)

(72) Inventors: Khareedu Venkateswara Rao, Hyderabad (IN); Vudem Dashavantha Reddy, Hyderabad (IN)

(73) Assignee: OSMANIA UNIVERSITY, Andhra Pradesh (IN)

(21) Appl. No.: 13/695,741

(22) PCT Filed: Nov. 25, 2010
(86) PCT No.: PCT/IB10/55408

§ 371 Date: Nov. 1, 2012

(30) Foreign Application Priority Data
Oct. 5, 2010 (IN) 2942/CHE/2010

(51) Int. Cl. A01H 5/00; C12N 15/82

(52) U.S. Cl. 800/289; 536/23.6; 435/320.1; 435/468; 800/278; 800/298; 435/419

(57) ABSTRACT

The present disclosure relates to identifying and characterizing polynucleotide sequences encoding proteins more particularly from Cajanus cajan, that are associated with abiotic stress responses in plants. In particular, the present disclosure provides a method for producing abiotic stress tolerant transgenic plant, more specifically salt, drought, heat and/or cold stress tolerant plant.

### TECHNICAL FIELD

The present disclosure relates to polynucleotide sequences encoding proteins that are associated with abiotic stress responses and abiotic stress tolerance in plants. In particular, the disclosure provides a method of obtaining abiotic stress tolerant plant.

### BACKGROUND

Survival, growth and yield potential of diverse crop plants are adversely impacted by rapid changes in environmental conditions caused by global warming. Abiotic stresses act as primary cause of crop yield losses worldwide, and pose a major threat to the sustainable food production as they reduce the potential yields of various crop plants by ˜50-70%. Plants often respond and adapt to the rapid climate changes through modulation of various physiological and molecular mechanisms. Stress is perceived and transmitted through signal transduction which affects regulatory elements of stress-inducible genes involved in the synthesis and/or alteration of different classes of proteins, viz., transcription factors, enzymes, molecular chaperones, ion channels, transporters, etc., resulting in stress tolerance. Molecular genetic and genomic tools have facilitated the identification of both functional and regulatory genes, while transformation methods have enabled genetic engineering of plants for production of abiotic stress tolerant crops. A clear understanding of the functions of stress-inducible genes also helps in unraveling the underlying mechanisms of stress tolerance. Functional genomic approaches, such as subtractive hybridization, differential screening, differential display, microarray analyses, reverse genetics, etc., have been employed to identify and define the functionality of various stress inducible genes.

Pigeon pea (Cajanus cajan L.) Millsp (2n=22) is a major grain legume of the arid and semi-arid regions of the world. This crop can be grown in a wide variety of soil textures ranging from sandy to heavy clays, and is usually cultivated under rain-fed conditions in hot-humid climates (Keller and Ludlow, J Exp Bot, 1993, 44:1351-59). Abiotic stresses exerted by drought, salinity, extreme temperatures, chemical toxicity, oxidative stress, etc., act as major impediments and pose a serious threat to the growth and productivity of crop plants. It has been estimated that 50% of the yield potential of major crops is routinely lost owing to the damages caused by these stresses. Among abiotic stresses, drought is the predominant factor that affects diverse plant functions leading to drastic decline in the crop productivity. Plants experience drought stress when the water supply to roots becomes scarce or when the transpiration rate is high. However, the general effects of drought stress on plant growth and the effects of water-deficit at biochemical and molecular levels are not well understood. In various crop plants, abiotic stress tolerance has been found to be complex and multigenic in nature. As such, unraveling the networks of interconnected pathways is essential to know about the responses of various stress-inducible genes. A clear understanding of the functions of stress-responsive genes also helps in analyzing the underlying mechanisms of stress tolerance.

In the recent past, significant contributions have been made in the isolation, cloning and characterization of different stress-inducible genes, as well as genetic engineering for stress tolerance in major crop plants. In model plant systems, isolation of various transcription factors, which mediates stress signaling, has become feasible.

Despite the availability of information on the molecular mechanisms of stress tolerance in model plants, additional investigations are needed in major crops for understanding and improving their stress tolerance. Since pigeon pea is a well known drought tolerant crop plant with profuse, deep-root system, it has been chosen as a source for cloning of stress inducible genes involved in abiotic stress tolerance.

### STATEMENT OF THE DISCLOSURE

Accordingly, the present disclosure is in relation to polynucleotide sequences as set forth in SEQ ID NO: 1 and SEQ ID NO: 2; polypeptide sequences as set forth in SEQ ID NO: 3 and SEQ ID NO: 4; a vector comprising polynucleotide sequence as set forth in SEQ ID NO: 1 or SEQ ID NO: 2 or a combination thereof; a recombinant cell comprising a vector as claimed in claim 6; a method of obtaining a recombinant cell, said method comprising acts of: a) inserting polynucleotide sequence as set forth in SEQ ID NO: 1 or SEQ ID NO: 2 or a combination thereof in a vector, and b) transforming a cell with the vector having said sequence to obtain the recombinant cell; a method of obtaining a transgenic plant comprising a polynucleotide sequence as set forth in SEQ ID NO: 1 or SEQ ID NO: 2 or a combination thereof, said method comprising acts of: a) obtaining a recombinant cell by method as claimed in claim 11, and b) inserting the recombinant cell into plant and culturing the plant to obtain the transgenic plant.

or comprising acts of: a) transforming a plant with a vector as claimed in claim 6, and b) culturing the transformed plant to obtain the transgenic plant; and a transgenic plant or plant part comprising polynucleotide sequences as set forth in SEQ ID No. 1 or SEQ ID No. 2 or polypeptide sequences as set forth in SEQ ID No. 3 or SEQ ID No. 4 or combinations thereof.

### BRIEF DESCRIPTION OF THE FIGURES

In order that the disclosure may be readily understood and put into practical effect, reference will now be made to exemplary embodiments as illustrated with reference to the accompanying figures. The figure together with a detailed description below, are incorporated in and form part of the specification, and serve to further illustrate the embodiments and explain various principles and advantages, in accordance with the present disclosure where:

FIG. 1: Shows comparison of deduced amino acid sequence of CcCDR of Pigeon pea with other closely related plant proteins.

FIG. 2: Shows effect of Pigeon pea cold and drought regulatory protein (CcCDR) in yeast against different abiotic stresses.

FIG. 3: Restriction map of T-DNA region of pBII21 containing CcCDR expression units with CaMV35S and rd29A promoters

FIGS. 4 and 5: Shows evaluation of CcCDR transgenic Arabidopsis plants against salt and cold stresses.

FIG. 6: Shows effect of CcCDR protein in transgenic tobacco plants subjected to drought, salt and cold stresses

FIG. 7: Shows CcCDR-transgenic plants subjected to abiotic stress conditions

FIG. 8: (a,b,c &d) Shows survival rate, plant biomass, root length and chlorophyll content of CcCYP Arabidopsis transgenic plants under mannitol, NaCl and cold stresses

FIG. 9: Shows survival rate, plant biomass, root length and chlorophyll content of CcCYP tobacco transgenic plants under mannitol, NaCl and cold stresses

FIG. 10: Shows estimation of proline and reducing sugars in CcCDR transgenic tobacco plants under different abiotic stresses.

FIG. 11: Shows comparison of the deduced amino acid sequences of Cajanus cajan cyclophilin (CcCYP) with CYPs from other species.

FIG. 12: Shows Northern blot analysis of Cajanus cajan cyclophilin (CcCYP) in Pigeon pea under different abiotic stress conditions. (a) Four-week-old Pigeon pea plants subjected to different concentrations of PEG and NaCl; (b) heat stress (37 and 42° C.); and (c) cold stress (4° C.).

FIG. 13: Shows structure of the T-DNA region of pBII21 containing Cajanus cajan cyclophilin (CcCYP), npt-II and gusA expression units and expression pattern of CcCYP in transgenic Arabidopsis plants. (a) Restriction map of the CcCYP expression cassette used for Arabidopsis transformation. The CcCYP gene is driven by the cauliflower mosaic virus 35S promoter. Nos. (nos terminator); RB (right) and LB (left) borders of T-DNA. (b) Northern blot analysis of CcCYP expression in control and transgenics (CcCYP) of Arabidopsis plants. Each lane was loaded with 10 mg of total RNA. C represents vector-transformed Arabidopsis, and CC1-CC4 represent four independent CcCYP transgenic lines of Arabidopsis.

FIG. 14: Shows effect of Cajanus cajan cyclophilin (CcCYP) protein in transgenic Arabidopsis plants subjected to drought, salt, heat and cold stresses. (a) Two-week-old seedlings of control and transgenics subjected to 300 mm mannitol for 7 d; (b) 100 mm NaCl for 7 d; (c) 37° C. for 90 min (pre-treatment) followed by 42° C. for 2 h; and (d) cold (4° C.) stress for 7 d. Photographs of seedlings were taken 10 d after recovery. C represents control; CC2 and CC4 represent two independent CcCYP transgenic lines.

FIG. 15: Shows effect of mannitol, NaCl, heat and cold stresses on control and Cajanus cajan cyclophilin (CcCYP) transgenics of Arabidopsis. Two-week-old seedlings of control and CcCYP transgenics were grown on 300 mm mannitol, 100 mm NaCl and cold stress (4° C.) for 7 d; for heat stress, seedlings were subjected to 37° C. for 90 min (pre-treatment) followed by 42° C. for 2 h. Seedlings were allowed to recover on MS plates. Data on (a) survival rate, (b) total biomass and (c) root length were recorded after 15 d of recovery. (d) Chlorophyll content was determined from the leaf discs of control and CcCYP transgenics after 72 h of incubation in 0, 300 mm mannitol and 100 mm NaCl solutions independently at room temperature (28±2° C.); for heat and cold stress, leaf discs were incubated in water at 42 and 4° C., respectively. Bar represents mean, and I represents SE from three independent experiments. ***, ** and * indicate significant differences in comparison with the control at P<0.001, P<0.01 and P<0.1, respectively. WS represents without stress; CC2 and CC4 represent two independent CcCYP transgenic lines; FW represents fresh weight.

FIG. 16: Shows evaluation of Cajanus cajan cyclophilin (CcCYP) transgenics against different abiotic stress conditions. Two-week-old seedlings of control and CcCYP transgenics were subjected to 300 mm mannitol (drought), 100 mm NaCl (salt) for 7 d; heat treatments were given at 37° C. for 11/2 h (pre-treatment) followed by 42° C. for 2 h. Treated seedlings were allowed to recover for 7 d at normal (20±1° C.) temperature. Later, seedlings from the plates were transferred to pots and allowed to grow for 3 weeks under normal conditions, and were photographed. C represents control; and CC2 and CC4 represent two independent CcCYP transgenic lines.

FIG. 17: Shows estimation of peptidyl-propyl cis-trans isomerase (PPIase) activity and its inhibition in control and Cajanus cajan cyclophilin (CcCYP) transgenic Arabidopsis lines. (a) PPIase-specific activity in control and transgenics. (b). Inhibition of PPIase-specific activity in the presence of cyclosporine A (CsA) inhibitor. Three-week-old unstressed and stressed (300 mm mannitol/100 mm NaCl) transgenic and control plants were used for extraction of total proteins. The PPIase activity was measured in a coupled assay using chymotrypsin (50 mg mL⁻¹), and change in the absorbance at 390 nm was monitored for 300 s. For inhibition of PPIase activity, 60 mm CsA was added to the reaction. Bar represents mean, and I represents SE from three independent experiments. ** indicates significant differences in comparison with the control at P<0.01, respectively. CC2 and CC4 represent two independent CcCYP transgenic lines.

FIG. 18: Estimation of Na⁺ ion content in control and Cajanus cajan cyclophilin (CcCYP) transgenic plants of Arabidopsis. Na⁺ ion levels in roots and shoots of control and transgenic plants were estimated after subjecting them to 100 mm NaCl treatment for 5 d. Bar represents mean, and I represents SE from three independent experiments. *** and ** indicate significant differences in comparison with the control at P<0.001 and P<0.01, respectively. CC2 and CC4 represent two independent CcCYP transgenic lines; DW represents dry weight.

FIG. 19: Shows subcellular localization of the transiently expressed Cajanus cajan cyclophilin (CcCYP):GFP fusion protein in onion epidermal cell as observed under confocal laser scanning microscope. Onion epidermal cells corresponding to GFP alone and CcCYP:GFP protein under bright (a,b) and fluorescence (c,d) field.=20 mm. N represents nucleus.

FIG. 20: Shows relative expression of AtSOS1 gene transcripts in shoot and root of Cajanus cajan cyclophilin (CcCYP) transgenics and control plants of Arabidopsis under salt and unstressed conditions. Bar represents mean, and I represents SE from two independent experiments. * indicates significant differences in comparison with the control at P<0.1. CC2 and CC4 represent two independent CcCYP transgenic lines; WS represents without stress.

### DETAILED DESCRIPTION OF THE DISCLOSURE

The present disclosure relates to polynucleotide sequences as set forth in SEQ ID NO: 1 and SEQ ID NO: 2.

The present disclosure also relates to polypeptide sequences as set forth in SEQ ID NO: 3 and SEQ ID NO: 4.

In an embodiment of the disclosure, the SEQ ID NOs: 3 and 4 corresponds to the polynucleotide sequence set forth in the SEQ ID NOs: 1 and 2 respectively.

In another embodiment of the disclosure, the sequences are obtained from plant species Cajanus.

In yet another embodiment of the disclosure, the sequences impart abiotic stress tolerance in species selected from a group comprising Arabidopsis species and Tobacco species.

The present disclosure also relates to a vector comprising polynucleotide sequence as set forth in SEQ ID NO: 1 or SEQ ID NO: 2 or a combination thereof.

In an embodiment of the disclosure, the vector is selected from a group comprising Agrobacterium based vector and E. coli based vector.

In another embodiment of the disclosure, the vector comprises an antibiotic selection marker.

The present disclosure also relates to a recombinant cell comprising a vector as claimed in claim 6.

In an embodiment of the disclosure, the cell is selected from a group comprising eukaryotic cells and prokaryotic cells.

The present disclosure also relates to a method of obtaining a recombinant cell, said method comprising acts of:

a) inserting polynucleotide sequence as set forth in SEQ ID NO: 1 or SEQ ID NO: 2 or a combination thereof in a vector; and
b) transforming a cell with the vector having said sequence to obtain the recombinant cell.

In an embodiment of the disclosure, the vector is selected from a group comprising Agrobacterium based vector and E. coli based vector; the cell is selected from a group comprising eukaryotic cells and prokaryotic cells; and the recombinant cell has abiotic stress tolerance.

The present disclosure also relates to a method of obtaining a transgenic plant comprising a polynucleotide sequence as set forth in SEQ ID NO: 1 or SEQ ID NO: 2 or a combination thereof, said method comprising acts of:

a) obtaining a recombinant cell by method as claimed in claim 11; and
b) inserting the recombinant cell into plant and culturing the plant to obtain the transgenic plant.
or comprising acts of:
   a) transforming a plant with a vector as claimed in claim 6;
   b) culturing the transformed plant to obtain the transgenic plant.

In an embodiment of the disclosure, the transgenic plant has abiotic stress tolerance.

In another embodiment of the disclosure, the abiotic stress is selected from a group comprising high temperature, low temperature, drought, salinity, oxidative stress, osmotic stress, chemical agents and any combination thereof.

In yet another embodiment of the disclosure, the high temperature is ranging from about 40° C. to about 46° C. preferably about 42° C.; the low temperature is ranging from about 4° C. to about 12° C. preferably about 4° C.; the salinity is ranging from about 0.1M to about 1M preferably about 0.2M; the chemical agents are selected from a group comprising polyethylene glycol (PEG), mannitol and combination thereof.

In still another embodiment of the disclosure, the polyethylene glycol (PEG) is at a concentration ranging from about 5% to about 25%, preferably about 20%, and the mannitol is ranging from about 100 mM to about 500 mM preferably about 300 mM.

The present disclosure also relates to a transgenic plant or plant part comprising polynucleotide sequences as set forth in SEQ ID No. 1 or SEQ ID No. 2 or polypeptide sequences as set forth in SEQ ID No. 3 or SEQ ID No. 4 or combinations thereof.

In an embodiment of the disclosure, the plant is selected from a group comprising Arabidopsis species and Tobacco species.

In another embodiment of the disclosure, the plant part is selected from a group comprising plant cell, seed, shoot, root, leaf, flower and fruit.

In yet another embodiment of the disclosure, the transgenic plant or plant part posses abiotic stress tolerance.

In still another embodiment of the disclosure, the abiotic stress is selected from a group comprising high temperature, low temperature, drought, salinity, oxidative stress, osmotic stress, chemical agents and any combination thereof.

In still another embodiment of the disclosure, the high temperature is ranging from about 40° C. to about 46° C. preferably about 42° C.; the low temperature is ranging from about 4° C. to about 12° C. preferably about 4° C.; the salinity is ranging from about 0.1M to about 1M preferably about 0.2M; the chemical agents are selected from a group comprising polyethylene glycol (PEG), mannitol and combination thereof.

In still another embodiment of the disclosure, the polyethylene glycol (PEG) is at a concentration ranging from about 5% to about 25%, preferably about 20%, and the mannitol is ranging from about 100 mM to about 500 mM preferably about 300 mM.

In an embodiment of the disclosure, Pigeon pea (Cajanus cajan L.) is a major grain legume crop of the semi-arid tropics, and is endowed with a substantial ability to withstand drought stress conditions. It grows well in hot, humid climates, and has an excellent deep root system with profuse laterals that facilitate extraction of moisture during drought periods. Identification of genes involved in environmental stress response offers scope for genetic engineering of crop plants for enhanced tolerance against abiotic stresses In this investigation, subtractive hybridization technique has been adopted to isolate different drought responsive genes from pigeon pea plants. Genes coding for Cyclophilin (CcCYP) and Cold and drought regulatory gene (CcCDR)—induced by various abiotic stresses—has been isolated and characterized. Over-expression of these genes in Arabidopsis plants afforded marked tolerance against major abiotic stresses.

The disclosure provides information on isolating and characterizing the genes expressed under drought-stress conditions in Pigeon pea plants. Two cDNA libraries of pigeon pea, in response to drought (PEG/water deficit) stress, have been constructed, and the functionality of drought-stress-induced ESTs has been annotated. Furthermore, selected genes associated with drought stress have been over-expressed in A. thaliana to verify their role in conferring abiotic stress tolerance. Arabidopsis plants expressing Pigeon pea genes conveyed high-level tolerance, enhanced plant biomass and increased photosynthetic rates under different abiotic stress conditions.

In an embodiment of the disclosure, Pigeon pea seeds of a highly drought tolerant variety, ICP 8744 were obtained from ICRISAT, Hyderabad, India and used as a source for the isolation of stress-inducible genes. Seeds were surface-sterilized with 0.1% mercuric chloride for 5 min followed by three washes, each for 10 min, in sterile distilled water under aseptic conditions. The sterilized seeds were soaked overnight in sterile water. Later, the swollen seeds were germinated on filter paper wetted with water in a tray and kept in the dark for 3-4 days. The germinated seedlings were grown either in Hoagland's nutrient solution or in pots containing soil, and were maintained in the glass house under controlled conditions at 28±2° C. and ˜70% humidity.

In an embodiment of the disclosure, for construction of cDNA libraries, 4-week old Pigeon pea plants, grown in the glass house, were treated independently with 10% polyethylene glycol (PEG-6000) for 6 h or subjected to water stress (sans watering) for 4 days. For transcript profiling of selected ESTs, Pigeon pea plants treated with PEG (10%) for 6 h/water stress (for 4 days)/salt stress (0.15 M for 6 h)/heat stress (42° C. for 4 h)/cold stress (4° C. for 24 h) were utilized. Water potential of control (unstressed) and stressed plants was measured adopting the pressure chamber method (Scholander et al. 1964).

In an embodiment of the disclosure, total RNA was isolated from stressed and unstressed Pigeon pea plants by guanidinium thiocyanate method (Sambrook and Russell, 2001), using 1.0 g of root and leaf tissues. The RNA pellet was washed with 70% ethanol, dried and dissolved in RNase-free water, and the amount of RNA was quantified by measuring the absorbance at 260 nm using a spectrophotometer.

An embodiment of the present disclosure relates to two subtracted cDNA libraries which were constructed using PCR-select-cDNA subtraction and cDNA-subtractive hybridization methods. PCR-select-cDNA subtractive hybridization was done according to the manufacturer's instructions (Clontech). The cDNA was synthesized from the mRNA of control and stressed Pigeon pea plants. The subtraction technique involves two hybridizations followed by suppression PCR. In the first hybridization, the denatured driver cDNA (control) was hybridized with two different tester (stressed) cDNA molecules (tester 1 and tester 2) separately, which were ligated with two different adaptors at the 5′ end of the double-strand cDNA. In the second hybridization, denatured driver cDNA (control) was mixed with two cDNA samples of the first hybridization. Later, using adaptor-specific primers, PCR was carried out followed by secondary PCR employing nested primers. The amplified products were ligated into pGEM T easy vector) and transformed into TOP10 cells of E. coli. From recombinant clones, plasmid DNA was isolated and analyzed by restriction enzyme digestions.

In an embodiment of the disclosure, subtractive hybridization was carried out and the mRNA was made from the total RNA isolated from control and stressed tissues of Pigeon pea using poly A tract mRNA isolation system III (Promega). For each subtraction, about 300-350 μg of stressed (tester) and 600-650 μg of control (driver) RNAs were taken. First strand cDNA was synthesized from driver mRNA using biotin oligo dT primer (Promega) and superscript reverse transcriptase. Driver cDNA and tester mRNA were hybridized at 65° C. for 1 h in buffer containing 0.5 M KCl and 10 mM Tris. Hybrids formed between tester mRNA and driver cDNA and excess first strand driver cDNA were separated from the unhybridized stressed mRNA using streptavidin paramagnetic particles (SPMPs, Promega). The unhybridized tester mRNA obtained was made into cDNA library using Stratagene ZAP-cDNA synthesis kit.

The Biological material present in the instant disclosure in the form of host cells comprising genetically modified vectors and the vectors comprising the genes of interest were deposited at the International Depository—Microbial Type Culture Collection & Gene Bank, Chandigarh. The deposited host cell/vector was assigned the following MTCC Numbers:

1) For CcCDR-MTCC 5594 (deposited on 27 Oct. 2010)
2) For CcCYP-MTCC 5595 (deposited on 27 Oct. 2010)

The present disclosure is elaborated by the following examples and figures. However, these examples should not be construed to limit the scope of the invention.

### Example 1

### Isolation and Cloning of Pigeon Pea (Cajanus Cajan L) Cold and Drought Regulatory Gene (CcCDR) Conferring Abiotic Stress Tolerance

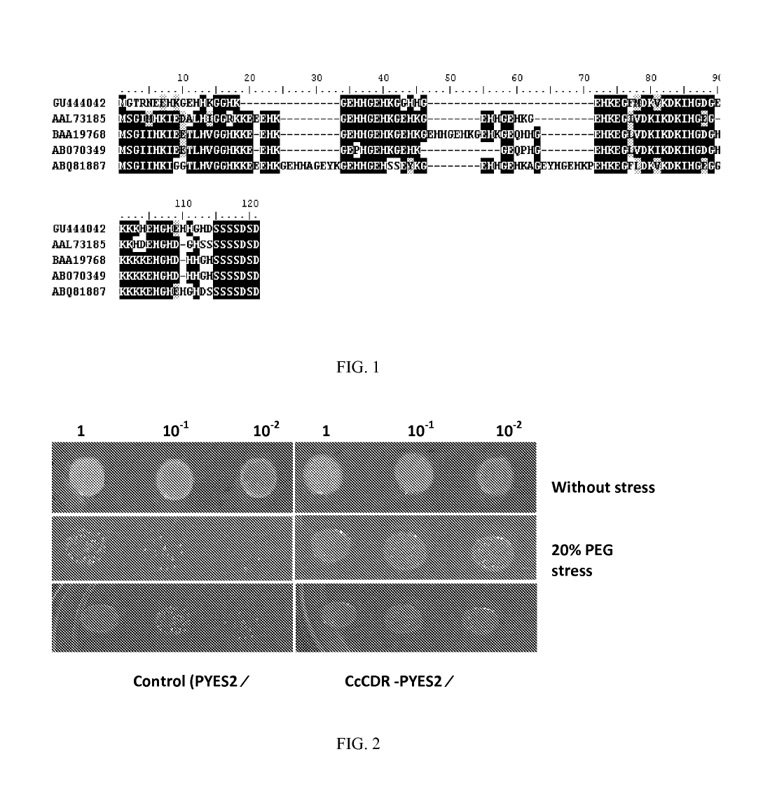

A full-length cDNA clone of 870 bp, with 5′ and 3′ untranslated regions, was obtained from the cDNA library of Pigeon pea plants subjected to 20% PEG stress (−1.01±0.02 Mpa) employing PCR-based cDNA subtraction. The clone (GU444042) contained the 282 bp coding sequence that codes for a polypeptide of 93 amino acids, and has been designated as Cajanus cajan cold and drought regulated gene (CcCDR). The CcCDR showed high identity of >73% with Carica papay (AAL73185; maturation associated like Srcl protein), >70% with Glycine max (BAA19768; Srcl), 68% with Glycine max (ABO70349; low temperature inducible protein), and >59% similarity with Glycine max (ABQ81887; KS-type dehydrin) (FIG. 1). To assess the nature of CcCDR, the genomic DNA was digested independently with BamHI, EcoRI, HindIII and SalI enzymes, and probed with the CcCDR coding sequence. For restriction digestion, 5 μl (2 μg) of plasmid DNA, 2 μl of 10× buffer, 0.5 μl of enzyme (1.5 U) and 12.5 μl of water were added in a reaction volume of 20 μl into a micro-centrifuge tube. The reaction mix was incubated at 37° C. for 3 hrs.

Southern analysis revealed single hybridization signals of varied size ranging from >2 Kb to 10 Kb. To investigate the stress-inducible nature of CcCDR, northern blot analysis was done using the RNA isolated from the plants treated with PEG (10% and 20%)/NaCl (1M)/cold (4° C.) along with untreated plants. Increased accumulations of CcCDR transcripts were detected in the stressed plants compared to the unstressed controls.

### Example 2

### Expression of CcCDR in Yeast Confers Abiotic Stress Tolerance

Yeast system was used to assess the effects of CcCDR protein against drought and salinity stress conditions. Yeast cells containing CcCDR under the regulation of GAL promoter expressed a polypeptide of ˜12 kD which was absent in the yeast transformed with the vector (pYES2/NT C) alone. Yeast strain harbouring pYES2/NTC-CcCDR along with the control (pYES2/NT C) was subjected to stresses induced by PEG and NaCl. Under normal (stress-free) conditions, the growth pattern of pYES2 NTC-CcCDR yeast was found similar to that of control yeast containing the vector alone (FIG. 2), Yeast cells expressing CcCDR showed normal growth under 20% PEG/1.0 mM NaCl stress compared to the negligible growth observed in the control yeast (FIG. 2) Also, CcCDR expressing yeast cells, when grown under similar stress conditions in the liquid medium, exhibited significant increases in growth rates as compared to the control yeast which showed negligible/no growth as indicated by OD₆₀₀ values (FIG. 2).

### Example 3

### Development of CcCDR Transgenics in A. Thaliana and Tobacco

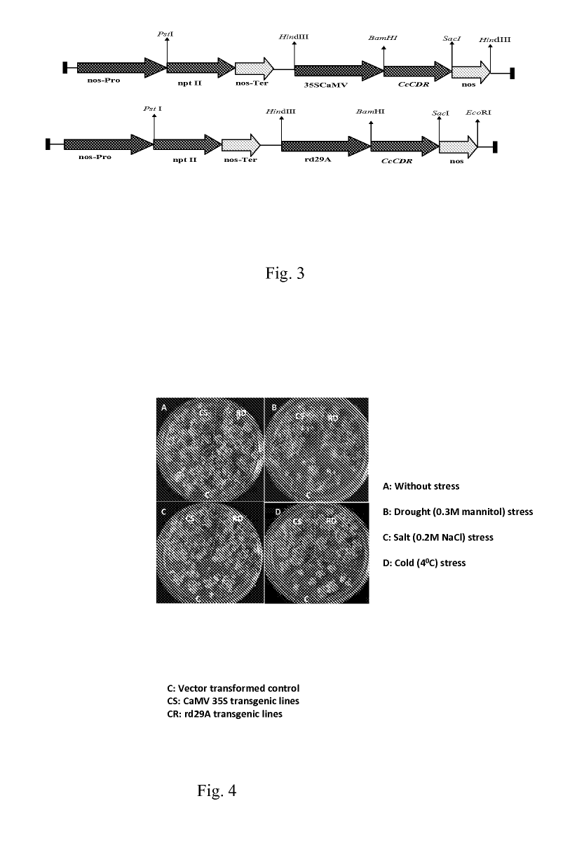

To investigate the role of CcCDR gene against abiotic stress, Arabidopsis and tobacco were transformed with CcCDR gene driven by CaMV 35S/rd29A promoters (FIG. 3).

### Mode of Transformation:

Coding region of CcCDR gene was cloned into pBII21 of Agrobacterium plasmid at BamHI and SacI restriction sites, driven by either CaMV 35S or rd29A promoter. The pBII21 vector containing CcCDR and nptII expression units were mobilized into EHA105 strain of Agrobacterium through triparental mating. Transformation of A. thaliana was carried out using Agrobacterium mediated vacuum infiltration method. Transformed seedlings were selected on MS medium supplemented with kanamycin (50 mg/L).

Transformation of tobacco plants (Nicotiana tabacum l.) cv was done using Agrobacterium mediated leaf disc method of transformation. (Horsch et al., 1988). Transformed seedlings were selected on MS medium supplemented with kanamycin (200 mg/L).

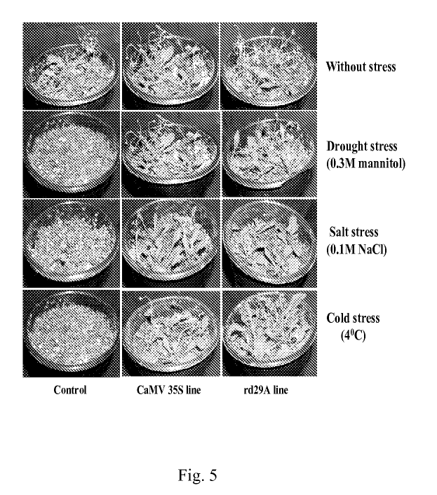

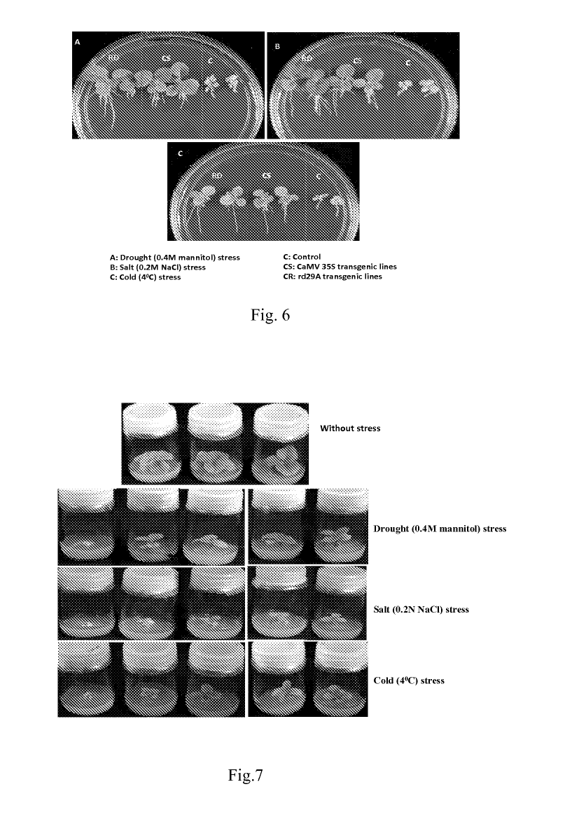

PCR analysis of the DNA isolated from the kanamycin tolerant Arabidopsis/tobacco plants, using CcCDR primers, revealed a ˜300 bp amplified fragment, while no such band was observed in the control plants. The presence of CcCDR transcripts was monitored by RT-PCR using the total RNA isolated from stressed rd29A-CcCDR transgenic lines (subjected mannitol, salt and cold), and unstressed CaMV35S-CcCDR transgenic lines as well as control plants; employing CcCDR-specific primers in both Arabidopsis and tobacco plants. Four independent homozygous (T3) lines of Arabidopsis viz., ACS1, ACS2 (CaMV35S-CcCDR) and ACR1, ACR2 (rd29A-CcCDR), and tobacco lines viz., TCS1, TCS2 (CaMV35S-CcCDR) and TCR1, TCR2 (rd29-A-CcCDR), were chosen for subsequent stress tolerance studies (FIGS. 4-7).

### Example 4

### Over-Expression of CcCDR in Arabidopsis Results in Enhanced Drought, Salt and Cold Tolerance

To evaluate the stress tolerance nature of CcCDR transgenics, 15-day old seedlings were subjected to 300 mM mannitol (drought stress) and 200 mM NaCl (salt stress) for seven days along with vector containing seedlings. After stress treatments, plants were allowed to recoup without stress for 15 d under normal conditions. Both ACS and ACR transgenic lines, when subjected to drought stress, showed higher survival rates of 80% and 90% as compared to control (48%) plants (FIG. 8.) Also, the transgenic lines showed disclosed substantial increases in the total biomass of 170% and 200%, as compared to control plants, under same stress conditions (FIG. 8). Similarly, the Arabidopsis transgenic lines (ACS and ACR), subjected to salt stress, showed higher survival rates of 82% and 91% and total biomass of 130% and 155% than that of control plants under similar conditions (FIG. 8.) Likewise, as compared to control plants the CcCDR transgenics (ACS and ACR lines) also displayed marked increases in root growth of 95% and 120% under 300 mM mannitol and 85% and 100% in 200 mM NaCl stress conditions (FIG. 8).

For analyzing the impact of CcCDR protein against cold stress, Arabidopsis transgenic plants were subjected to cold (4° C.) stress. Under cold stress, transgenic lines (ACS and ACR) showed distinct increases in the total biomass of 120% and 140% compared to control plants (FIG. 8). Moreover, the transgenic lines also disclosed increased root growth of 70% and 90% under cold treatments (FIG. 8).

### Example 5

### Over-Expression of CcCDR in Tobacco Confers Enhanced Drought, Salt and Cold Tolerance

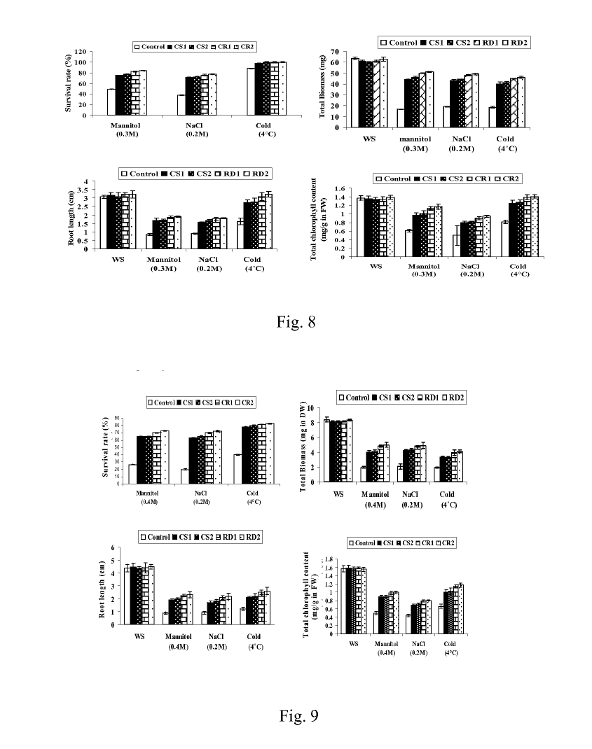

To evaluate the stress tolerance nature of CcCDR transgenic tobacco lines, 20-day old seedlings were subjected to 400 mM mannitol (drought stress) and 200 mM NaCl (salt stress) for 10 days along with the vector containing seedlings. After stress treatments, plants were allowed to recoup for 15 days under normal (without stress) conditions. Both TCS (TCS1, TCS2) and TCR (TCR1, TCR2) transgenic lines, when subjected to drought stress, showed higher survival rates of 65% and 80% compared to the control (27%) plants (FIG. 9). Further, these transgenics revealed substantial increases in the total biomass by 110% and 140% (FIG. 9). Similarly, the tobacco transgenic lines (TCS and TCR), when subjected to salt stress, showed higher survival rates of 65% and 75%, and total biomass of 105% and 130% than that of control plants under identical conditions (FIG. 9), Likewise, when compared to the control plants, the CcCDR transgenics displayed marked increases in the root growth of 120% and 150% under 400 mM mannitol, and 95% and 135% increases under 200 mM NaCl stress (FIG. 9.).

Tobacco CcCDR-transgenic lines subjected to cold (4° C.) stress showed higher survival rates of 80% and 83% as compared to the control plants (40%). These transgenic lines also showed marked increases in the total biomass of 72% and 110% when compared to the control plants (FIG. 9). Moreover, the transgenics also exhibited increased root growth of 80% (TCS) and 110% (TCR) under similar conditions (FIG. 9.).

### Example 6

### Effect of Abiotic Stress Treatments on Total Chlorophyll Content of CcCDR-Transgenics of Arabidopsis and Tobacco

The leaf disks of ACS (ACS1 & ACS2), ACR (ACR1 & ACR2) lines of Arabidopsis transgenic lines, and TCS (TCS1 & TCS2), TCR (TCR1 & TCR2) lines of tobacco along with control leaves were floated independently for 72 h on 0 mM, 300/400 mM mannitol (drought stress), 200 mM NaCl (salt stress) solutions, and also on water at 4° C. (cold stress). These leaf discs were used for measuring chlorophyll content spectrophotometrically after extraction in dimethlysulphoxide for 2 hours.

Transgenic plants subjected to mannitol stress revealed higher (60% and 100%) mean chlorophyll content as compared to control plants. Likewise, transgenic plants treated with NaCl disclosed higher (60% and 89%) chlorophyll contents as compared to the control plants. Furthermore, transgenic plants subjected to cold (4° C.) stress divulged substantially higher (54% and 77%) chlorophyll contents in the Arabidopsis and tobacco transgenic lines when compared to the control plants (FIGS. 8&9).

### Example 7

### Proline and Reducing Sugar Contents CcCDR Transgenic Tobacco Plants Under Stress Conditions

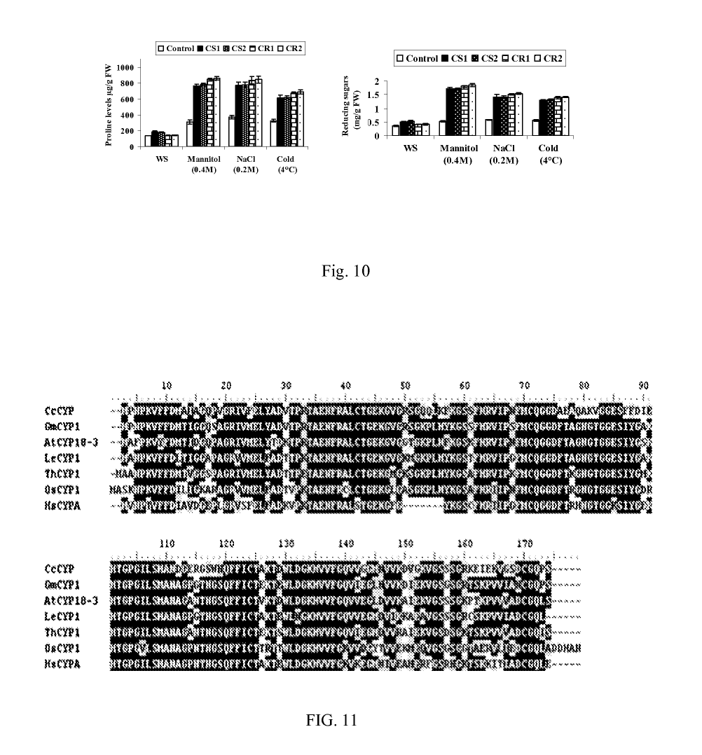

To understand the physiological basis for the improved stress tolerance of transgenic tobacco, proline and reducing sugars contents were estimated in tobacco plants expressing CcCDR gene under stress conditions as well as under normal growth conditions. Four CcCDR tobacco transgenic lines (TCS1, TCS2, TCR1, and TCR2) were subjected to 400 mM mannitol (drought stress), 200 mM NaCl (salt stress) and 4° C. (cold stress) for seven days along with the vector containing control seedlings. Both the transgenic lines (CaMV35S-CcCDR and rd29a-CcCDR), when subjected to drought (400 mM mannitol) stress, accumulated 147% to 179% higher contents of proline, and 240% to 250% higher contents of reducing sugars compared to the control plants (FIG. 10). Similarly, transgenic plants subjected to salt (200 mM NaCl) stress accumulated 110% to 125% higher contents of proline, and 144% to 160% higher contents of reducing sugars than that of control plants. Likewise, under cold (4° C.) stress, both the transgenic plants accumulated, approximately, 90% to 110% higher contents of proline, and 135% to 150% higher contents of reducing sugars compared to the control plants. Here, it should be noted that Proline was estimated according to Bates et al. (1973) method and reducing sugars were estimated using the 3,5-dinitrosalicylic acid method of H. Lindsay (1973).

Significant differences in proline (35%) and reducing sugars (40%) contents were also detected in transgenic lines of CaMV35S-CcCDR when compared to the control tobacco plants and rd29A-CcCDR transgenic plants under normal conditions (FIG. 10).

### Example 8

### Construction of Subtractive cDNA Library and Isolation of CcCYP from Pigeon Pea

Total RNA was isolated from the 4-week-old control and water-stressed Pigeon pea plants by guanidinium thiocynate (GTC) method. mRNA was isolated from the total RNA through biotin-labelled oligo (dT) probe using mRNA isolation kit (Promega, Madison, Wis., USA). cDNA library was constructed through subtractive hybridization using one part of poly (A)⁺ RNA from stressed (tester) plants and five parts of 5′-biotinylated first strand cDNA from unstressed (driver) plants. The poly (A)⁺ RNA-cDNA hybrids and the excessive cDNA were immobilized onto streptavidin-coated magnetic beads. The unbound subtracted poly (A)⁺ RNA was used to synthesize the first-strand followed by the second-strand cDNA. The cDNA fragments were ligated to a lambda-ZAP vector, in vitro packaged and allowed to infect XL1 blue MRF Escherichia coli cells as per the manufacturer's instructions, using a Uni-ZAP XR cDNA library construction kit (Stratagene, Lajolla, Calif., USA). Cloned cDNA fragments were sequenced independently with T7 and T3 promoters using automated DNA sequencer and nucleotide and amino acid sequences were analysed employing BLAST (NCBI) and ExPASy tools. Based on sequence analysis of the cDNA clone, it was designated as Cajanus cajan CYP gene (CcCYP). Multiple sequence alignment was performed employing CLUSTALW using Bioedit software.

A cDNA clone (GU 238312) coding for a CYP was obtained from the cDNA library of Pigeon pea plants subjected to water stress (50-60% RWC) by subtractive hybridization. The clone contained 519 bp coding sequence that codes for a polypeptide of 172 amino acids (aa) and has been designated as CcCYP gene. Amino acid sequence analysis of CcCYP protein divulged the presence of a single conserved CYP PPIase domain including R, F and H residues required for PPIase activity. The CcCYP showed high identity of >73% with Glycine max (GmCYP1), 67% with Lycopersicon esculentum (LeCYP1), >65% with A. thaliana (AtCYP18-3/ROC1), >65% with T. halophila (ThCYP1), >57% with Oryza sativa (OsCYP) and >59% with that of Homo sapiens (HsCYPA) (FIG. 11)

### Example 9

### Northern Blot Analysis for CcCYP Gene

Northern blot was carried out with 10-20 mg of total RNA isolated from Pigeon pea and Arabidopsis plants. Northern blot analysis was performed according to Sambrook & Russell (2001). The α-³²P-dCTP-labelled CcCYP cDNA was used as a probe, and hybridization was detected by autoradiography. Ethidium bromide-stained r-RNA bands were used to assess the quality and quantity of RNA.

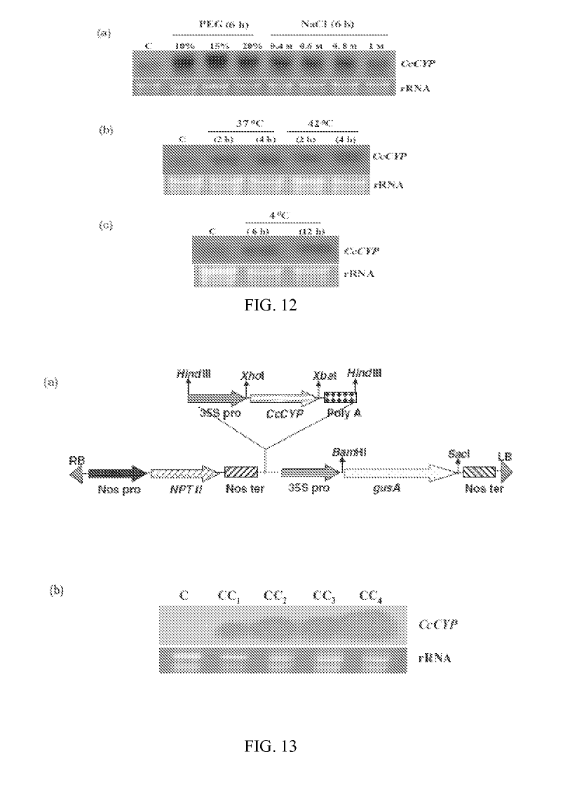

To examine the stress-inducible nature of CcCYP, Northern blot analysis was performed using the total RNA isolated from Pigeon pea plants treated with different concentrations of polyethylene glycol (PEG-10, 15 and 20%) and NaCl (0.4, 0.6, 0.8 and 1.0 m) for 6 h along with untreated plants. Increased levels of CcCYP transcripts were detected in PEG- and NaCl-treated plants as compared to untreated plants (FIG. 12a). Northern analysis of plants subjected to higher temperatures at 37 and 42° C., and cold stress at 4° C., revealed increased transcript levels of CcCYP when compared with the control plants grown at 28±2° C. (FIGS. 12b,c).

### Example 10

### Construction of Plant Expression Vector and Arabidopsis Transformation for CcCYP Gene

Full-length CcCYP (GU 238312) coding sequence was amplified with Pfu DNA polymerase using 5′-GCCTC GAGATGCCTAACCCTAAGGTTTT-3′ (forward, XhoI site underlined), 5′-GCTCTAGACTAAGAGGGTTGA CCGCAG-3′ (reverse, XbaI site underlined) primers. The CcCYP coding region was cloned into XhoI and XbaI sites of pRT100 plasmid in the sense orientation, and the expression unit (35S:CcCYP: PolyA) was excised with HindIII and cloned into the HindIII site of the pBII21 vector (Clontech, Mountain View, Calif., USA) containing gusA and nptII (kanamycin) expression units. The pBII21 and CcCYP constructs were then mobilized into Agrobacterium tumefaciens strain (EHA105) by triparental mating. Agrobacterium-mediated transformation was performed via the vacuum infiltration method of A. thaliana. Seeds were harvested from transformed plants, and plated on kanamycin (50 mg mL⁻¹) selection medium to identify the putative transgenic plants. Kanamycin-resistant T1 transgenic plants were screened for the presence of T-DNA by GUS staining of seedlings, and also confirmed by PCR analysis using gene-specific primers of gusA (5′-GGAAAAGTGTACGTATCACCGTTTG-3′ and 5′-TATCAGCTCTTTAATCGCCTGTAAG-3′) and CcCYP (as described above). PCR products were analysed on 0.8% (w/v) agarose gel containing ethidium bromide. Later, PCR products were blotted onto Hybond-N⁺ charged nylon membrane, and were hybridized with the gusA coding sequence labelled with α-³²P-dCTP (Sambrook & Russell 2001). Two transgenic lines of CC2 and CC4 (T3 generation) along with vector containing line (control) were selected for further stress tolerance studies.

To investigate the role of CcCYP against abiotic stress, the coding sequence of the gene was cloned downstream to CaMV 35S promoter in pBII21 vector containing nptII as a selectable marker along with gusA gene (FIG. 13a). Agrobacterium strain (EHA105) carrying pBII21-nptII-gusA (control) or pBII21 containing CcCYP, and nptII and gusA expression units were employed for transformation of A. thaliana. Transformed seedlings were selected on MS medium supplemented with kanamycin (50 mg mL⁻¹). PCR analysis of the genomic DNA of control, T1 and T2 transgenic plants, employing gusA gene-specific primers, revealed a >800 bp amplified fragment, while no such band was observed in the DNA of wild-type plants. When the genomic DNA of T1 and T2 transgenic plants were subjected to PCR with CcCYP gene-specific primers, they disclosed a >500 bp amplified fragment; however, no such band was observed in the vector (pBII21-nptII-gusA)-transformed plants. Southern analysis of PCR products obtained with gusA primers, when probed with gusA coding sequence, showed a hybridizable band of ˜800 bp in the control and transgenic plants. Furthermore, transformed lines of CcCYP- and vector-containing plants showed the expression of gusA as evidenced by intense blue colour. Northern analysis of four independent T3 transgenic lines showed varied levels of transgene expression (FIG. 13b). Transgenics CC2 and CC4 with distinctly higher levels of CcCYP transcripts were chosen for subsequent stress tolerance studies.

### Example 11

### Functional Analysis of CcCYP Transgenics for Abiotic Stress Tolerance

Seeds of the control and transgenic Arabidopsis were surface-sterilized and grown on MS salt medium (Murashige & Skoog 1962) or in soil (mixture of 1 vermiculite:1 perlite:lsoilrite), and were kept at 4° C. in dark for 3 d for stratification. Later, they were transferred to Conviron growth chamber (model TC16, Winnipeg, Manitoba, Canada) and were allowed to grow at 20±1° C. under long-day conditions (16 h light/8 h dark cycles) with fluorescent light (7000 lux at 20 cm). To test for drought and salt tolerance, 2-week-old seedlings were grown on MS medium supplemented with mannitol (0.3 m) or NaCl (0.1 m) for 1 week. To test the cold sensitivity, 2-week-old seedlings were transferred to incubator set at 4° C. for 7 d. For heat treatment, 2-week-old seedlings were exposed to 37° C. for 90 min (pre-treatment) followed by 42° C. for 2 h. After stress treatments, the seedlings were allowed to recover on MS medium under normal conditions (20±1° C., 16 h light/8 h dark cycles, 7000 lux at 20 cm) in the growth chamber, and survival rate, root length and biomass were recorded after 15 d of recovery. Hypocotyl elongation assay was performed by subjecting the germinated seedlings to 37° C. for 90 min, followed by 2 h of recovery under normal conditions, and were exposed to 42° C. for 2 h. Data were recorded on hypocotyl elongation after 3 d of recovery. All the experiments were replicated thrice using 20 seedlings per treatment

Two-week-old seedlings of the control and transgenics were grown on MS medium added with mannitol (0.3 m)/NaCl (0.1 m), or subjected to cold (4° C.), for 15 d, and seedling survival rate, total biomass and root length were recorded without any recovery period.

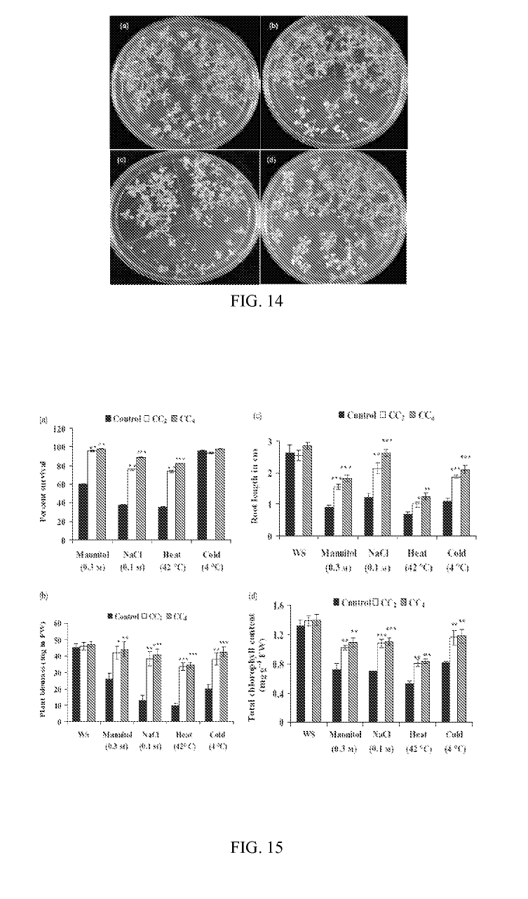

To evaluate the stress tolerance nature of CcCYP transgenics, 2-week-old seedlings were subjected to 300 mm mannitol (drought stress) and 100 mm NaCl (salt stress) for 7 d along with vector-containing seedlings. Both transgenic lines, when subjected to drought stress, showed higher survival rates of ˜95 and ˜97% as compared to the control (˜60%) plants (FIGS. 14a & 15a). These transgenics, compared to the control plants, disclosed substantial increases in the total biomass of ˜60 and ˜68% (FIG. 15b). Similarly, the transgenic lines, subjected to salt stress, showed higher survival rates of ˜75 and ˜88%, and total biomass of ˜119 and ˜216% than that of the control plants under similar conditions (FIGS. 14b & 15a,b). Likewise, as compared to the control plants, the CcCYP transgenics also displayed marked increases in root growth of ˜68 and ˜97% under 300 mm mannitol, and ˜76 and ˜114% in 100 mm NaCl stress (FIG. 15c).

The CcCYP transgenics (CC2 and CC4), upon exposure to high (42° C.) temperature, disclosed increased survival rates of ˜73 and ˜82% in comparison with ˜35% survival observed in the control plants (FIGS. 14c & 15a), and also revealed increased total biomass of ˜230 and ˜250% (FIG. 15b). In addition, notable increases of ˜44 and ˜84% were observed in the elongation of hypocotyls of both transgenics. The transgenic plants, when subjected to cold (4° C.) stress, showed distinct increases in the total biomass of ˜89 and ˜110% compared to the control plants (FIGS. 14d & 15b). Moreover, the transgenics also showed increased root growth under heat (˜50 and ˜80%) and cold (˜70 and ˜90%) treatments (FIG. 15c).

Two-week-old seedlings of transgenic lines, when subjected to 300 mm mannitol (drought stress)/100 mm NaCl (salt stress) for 15 d, showed higher survival rates of ˜75 to ˜82%, and ˜86 to ˜91%, respectively, as compared to the control (˜48%) plants. In addition, substantial increases in the total biomass of ˜110 to ˜122%, and ˜109 to ˜117% were observed in the transgenic lines under drought and salt stress when compared to the controls. Likewise, as compared to the control plants, the CcCYP transgenic lines revealed significant increases in root growth of ˜68 and ˜97% under 300 mm mannitol, and ˜105 and ˜120% in 100 mm NaCl treatment. The transgenic plants, subjected to cold (4° C.) stress for 15 d, showed survival rates of ˜98 and ˜100% compared to ˜88% in the control plants. In addition, significant increases were observed in the total biomass of the transgenic lines (˜80 and ˜88%) compared to that of the control plants. Similarly, the transgenics showed increased root growth of ˜38 and ˜65% compared to the control plants under cold stress.

Leaf discs of CC2 and CC4 transgenic lines, subjected to mannitol (300 mm) stress, revealed higher mean chlorophyll content of ˜43 and ˜53%, respectively, as compared to the control plants. Likewise, the transgenic plants treated with NaCl (100 mm) disclosed substantially higher chlorophyll content (˜56 and ˜59%) when compared to the control plants. Furthermore, the transgenic plants subjected to heat (42° C.) and cold (4° C.) treatments divulged increased (>50 and >40%) total chlorophyll contents in comparison with the control plants (FIG. 15d).

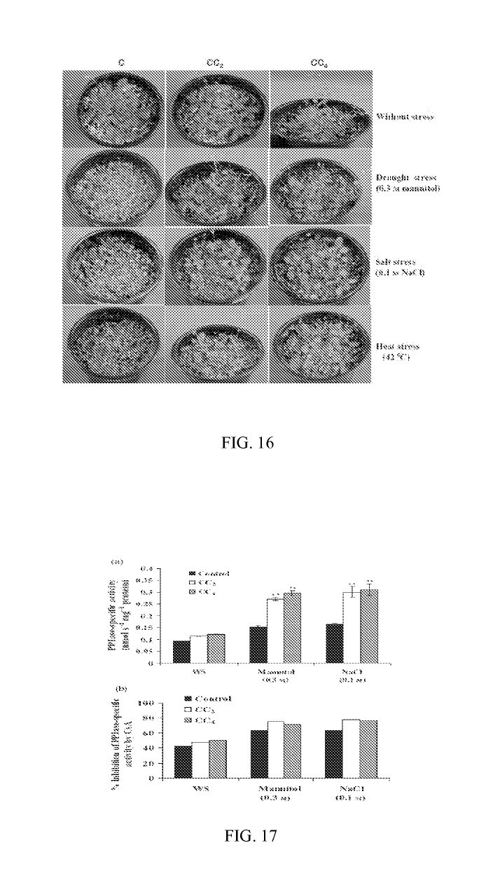

Furthermore, the transgenic plants expressing CcCYP could successfully complete their reproductive cycle, while the control plants (except under cold stress) turned chlorotic and failed to reach the reproductive phase under drought, salt and heat stress conditions (FIG. 16).

### Example 12

### Peptidyl Prolyl Cis-Trans Isomerase (PPIase) Assay in CcCYP Transgenics

Three-week-old transgenic and control plants treated with 0.05×MS salts containing 300 mm mannitol/100 mm NaCl for 3 d were used for extraction of total proteins as described (Lippuner et al. 1994 Journal of Biological Chemistry, 269, 7863-7868). Protein concentration of samples was determined as per the method of Bradford. PPIase activity was measured in a coupled assay with chymotrypsin as described by Breiman et al. [1992, Journal of Biological Chemistry, 267, 21293-21296] with certain modifications like use of 50 mM Hepes instead of 40 mM, absorbance was monitored for 300 sec instead of 100 sec and final reaction volume was made to 1 ml instead of 1.5 ml.

Test peptide N-succinyl-Ala-Ala-Pro-Phe-p-nitroanilidine at 60 mm final concentration was added to a solution of the assay buffer [50 mm HEPES (pH 8.0), 0.015% Triton X-100] and plant extract (300 mg) in a final volume of 1 mL. The reaction was initiated by adding chymotrypsin (50 mg mL−1), and change in the absorbance at 390 nm was monitored for 300 s. CYP-associated PPIase activity was determined by the extent of inhibition of reaction in the presence of CsA (60 mm). CsA inhibitor was added to the assay mix for 30 min before the start of the reaction, and incubated at 4° C. For calculating the PPIase activity, differences between the catalysed and uncatalysed first-order rate constants, derived from the kinetics of absorbance change at 390 nm, were multiplied with the amount of substrate in each reaction. The PPIase activity recorded in the control plants is deemed as theinnate activity (IPA). Transgene-specific PPIase activity (TPA) is derived by subtracting the activity of the unstressed control plants (IPA) from that of the activity of the unstressed transgenics. Transgene-induced PPIase activity (TIPA) is calculated by subtracting the activity of the stressed control (IPA) and transgene-specific activity (TPA) from the total PPIase activity of the stressed transgenics.

CcCYP-expressing transgenic lines, subjected to 0.3 m mannitol stress for 72 h, showed increased PPIase activity of ˜0.27 and ˜0.30 nmol s−1 mg−1 protein compared to ˜0.15 nmol s−1 mg−1 protein observed in the control plants under similar stress conditions. Likewise, the CcCYP transgenics grown under 0.1 m NaCl stress for 72 h also exhibited enhanced PPIase activity (˜0.30 and ˜0.31 nmol s−1 mg−1 protein) compared to the control plants (FIG. 17a). Under both stress conditions, the transgenic lines, compared to the control, showed additional PPIase activity of 0.095-0.126 nmol s⁻¹ mg⁻¹ protein (Table 1). However, no such additional activity was noticed in the control and transgenics under unstressed conditions. In the presence of CsA, the unstressed transgenic lines exhibited ˜47 and ˜50% inhibition of PPIase activity, while the control plants showed ˜42% inhibition. Whereas, under stressed conditions, the transgenic lines and control plants revealed >70 and ˜63% inhibition of PPIase activity, respectively (FIG. 17b).

TABLE 1
Peptidyl prolyl cis-trans isomerase (PPIase) activity (nmol s−1 mg−1 protein) pf Cajanus cyclophilin (CcCYP)
transgenic Arabidopsis lines under drought and salt stress.

| Control | | | | | | | | | |
|---|---|---|---|---|---|---|---|---|---|
| and | | | | | | | | | |
| trans- | | | | | | | | | |
| genic | | | | | | | | | |
| lines | Unstressed | | | Mannitol (0.3M) (drought stress) | | | NaCl (0.1M) (salt stress) | | |
| C | IPA | TPA | TIPA | IPA | TPA | TIPA | IPA | TPA | TIPA |
| | 0.093 ± 0.002 | — | — | 0.152 ± 0.006 | — | — | 0.164 ± 0.005 | — | — |
| CC₂ | 0.093 ± 0.002 | 0.024 ± 0.006 | — | 0.152 ± 0.006 | 0.024 ± 0.006 | 0.095 ± 0.006 | 0.164 ± 0.005 | 0.024 ± 0.006 | 0.118 ± 0.024 |
| CC₄ | 0.093 ± 0.002 | 0.030 ± 0.001 | — | 0.152 ± 0.006 | 0.030 ± 0.001 | 0.114 ± 0.007 | 0.164 ± 0.005 | 0.030 ± 0.001 | 0.126 ± 0.019 |

Values represent mean ± SE from three independent experiments.
IPA, innate PPIase activity,
TPA, inorgene-specific PPIase activity,
TIPA, transgene-induced PPIase activity.
C, control; CC₂ and CC₄, CcCYP transgenic lines

### Example 13

### Measurement of Chlorophyll Content Under Stress Treatments in CcCYP Transgenics

Leaf discs from 3-week-old transgenic and control plants were floated in a 20 mL solution of NaCl (100 mm)/mannitol (300 mm) or water (experimental control) for 72 h at room temperature (28±2° C.). For heat and cold stresses, leaf discs were floated in 20 mL of water and kept at 42/4° C. for 72 h. The treated leaf discs were then used for measuring chlorophyll spectrophotometrically after extraction in dimethylsulphoxide (DMSO) for 2 h.

### Example 14

### Measurement of Na⁺ Ion Content in CcCYP Transgenics

For measurement of sodium ion content, 3-week-old untreated control and transgenic plants, as well as plants treated with 0.05×MS salts containing 100 mm NaCl, for 5 d were used. Later, leaves and roots from untreated and treated plants were harvested separately, and dry weights of samples were recorded after thorough drying at 80° C. for 2 d. The samples were digested with HNO3, and Na+ ion concentration was assayed by atomic emission spectrometry (model GBCAAS932).

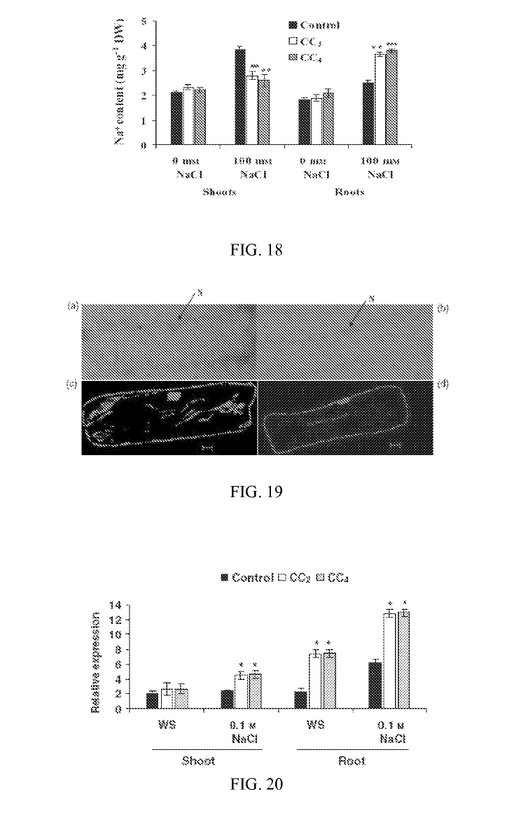

The roots of both transgenic lines, grown under salt stress (100 mm NaCl), accumulated higher levels of Na⁺ ions (3.6±0.09 and 3.9±0.09 mg g−1 dry weight) than that of the control (2.5±0.19 mg g⁻¹) plants (FIG. 18). Conversely, the control plants, compared to the transgenics (2.8±0.17 and 2.6±0.26 mg g⁻¹), accumulated higher levels of Na+ ions (3.8±0.12 mg g−1) in shoots when grown under similar stress conditions. However, under unstressed conditions, the roots and shoots of the transgenics and control plants accumulated low levels of Na+ ions exhibiting minor differences between them (FIG. 18).

### Example 15

### Subcellular Localization of CcCYP Protein

A cDNA fragment containing the Pigeon pea CcCYP ORF was fused with the 5′ end of the green fluorescent protein (gfp) coding region, and the fused product was subcloned into the pBII21 expression vector under the control of CaMV 35S promoter. The plasmid vector containing CaMV35S-gfp-nos was used as the control. Two micrograms of the plasmid construct was used to coat tungsten particles for transformation of onion epidermal cells. The epidermis was peeled off and carefully placed onto MS medium containing 2% agar. Epidermal peels were bombarded with plasmid-coated tungsten particles using a gene gun (Genepro, Hyderabad, India 2000He) with 1100 psi under a vacuum of 28 in. Hg and target distance of 6 cm. After bombardment, the epidermal peels were incubated at 25° C. for 24 h in the dark, and were then visualized using a laser scanning confocal microscope (TCS ST; Leica microsystem, Heidelberg, Germany).

To examine the subcellular localization of CcCYP protein, the CcCYP: gfp fusion and gfp (control) constructs were independently bombarded into the onion epidermal cells. Epidermal cells containing pBII21-gfp plasmid showed fluorescence throughout the cell owing to the expression of GFP in the cytoplasm and nucleus (FIG. 19c). However, the CcCYP: GFP fusion protein was found to fluoresce predominantly in the nucleus, while it was weak in the cytosol (FIG. 19d).

### Example 16

### Quantitative Real-time PCR (qRT-PCR)

qRT-PCR was performed for A. thaliana salt overly sensitive (AtSOS1) gene using oligonucleotide primers of 5′-CCAATGAAACTGCGTGGTG-3′ and 5′-GCACT TTCCTGCCAAAGG-3′. First-strand cDNA was synthesized from RNA samples of the control and transgenic Arabidopsis seedlings subjected to NaCl (0.1 m) stress for 7 d, as well as from unstressed plants. The resultant cDNAs were used as templates for qRT-PCR analysis. DNase treatment was given for removing contaminating genomic DNA from RNA samples. RT-PCR analysis was carried out using Eurogentec SYBR Green qPCR Master mix with Real-Plex4 (Eppendorf, Hamburg, Germany) at 94° C. (1 min), 58° C. (1 min) and 72° C. (1 min) for 30 cycles. Later, the products were analysed through a melt curve analysis to check the specificity of PCR amplification. Each reaction was performed twice, and the relative expression ratio was calculated using 2-DDCt method employing actin gene as reference. Oligonucleotide primers of 5′-GGCGATGAAG CTCAATCCAAACG-3′- and 5′-GGTCACGACCAGCAAGATCAAGACG-3′ were used for amplification of actin gene. Mean values, standard error and t-test were computed with the help of pre-loaded software in Excel, programmed for statistical calculations.

The transgenic Arabidopsis lines expressing CcCYP and control plants, subjected to 100 mm NaCl stress as well as unstressed conditions, were analyzed for the expression levels of AtSOS1 gene by using quantitative RT-PCR. Under unstressed conditions, the shoots of CcCYP transgenic plants showed increase (2.67) in the relative expression of AtSOS1 gene compared to that of the control plants (2.00). Similarly, under NaCl stress, the shoots of the transgenic plants revealed increased (4.62) AtSOS1 expression as compared to the control plants (2.30). The roots of the unstressed transgenic plants exhibited increased (7.46) relative expression of AtSOS1 compared to the control plants (2.25). Likewise, the roots of the transgenic plants under salt stress disclosed enhanced (12.99) AtSOS1 transcripts compared to the control plants (6.19). The roots of the transgenic and control plants, compared to the shoots, exhibited higher levels of AtSOS1 transcripts both under stressed and unstressed conditions (FIG. 20).

### Example 17

### Comparison of Parameters Between CcHyPRP, CcCYP and CcCDR Under Different Stress Conditions

The following comparative study illustrates the role of the two genes CcCYP and CcCDR in the Arabidopsis. The two genes of the present disclosure were compared with another gene from Pigeon Pea viz. CcHyPRP (Cajanus Cajan hybrid-proline-rich protein).

TABLE 2
Comparison of parameters between CcHyPRP, CcCYP and CcCDR
under different stress conditions

| | Cajanus cajan hybrid- | | Cajanus cajancold and |
|---|---|---|---|
| | proline-rich protein | Cajanus cajan | drought regulatory gene |
| Nature of stress | encoding gene (CcHyPRP) | cyclophilin gen (CcCYP) | (CcCDR) |
| DROUGHT | 85.00%-88.33% increase | ~97% increase in | 90% increase in survival |
| STRESS | in survival rate over wild- | survival rate over wild- | rate |
| [MANNITOL | type | type | over wild-type |
| (300 mM)] | 96.00%-144.50% | ~68% increase in | 170% to 200% increase in |
| | increase in biomass over | biomass over wild-type | biomass over wild-type |
| | wild-type | ~97% increase in root | 95% to 120% increase in |
| | 184.62%-215.30% | length over wild-type | root length over wild-type |
| | increase in root length | ~53% higher chlorophyll | 60% to 100% higher |
| | over wild-type | content over wild-type | chlorophyll |
| | | | content over wild-type |
| SALT STRESS | 85.00%-88.33% increase | ~88% increase in | 91% increase in survival |
| [SALT (200 mM)] | in survival rate over wild- | survival rate over wild- | rate over wild-type |
| | type | type | 130% to 155% increase in |
| | 524.60%-671.20% | ~216% increase in | biomass over wild-type |
| | increase in biomass over | biomass over wild-type | 85% to 100% increase in |
| | wild-type | ~114% increase in root | root length over wild-type |
| | 286.60%-420.00% | length over wild-type | 60% to 89% higher |
| | increase in root length | ~56 and ~59% higher | chlorophyll content over |
| | over wild-type | chlorophyll content over | wild-type |
| | | wild-type | |
| COLD STRESS | showed no observable | 97% increase in | 83% increase in survival |
| (4° C.) | differences | survival rate over wild- | rate over wild-type |
| | | type | 120% to 140% increase in |
| | | ~110% increase in | biomass over wild-type |
| | | biomass over wild-type | 70% to 90% increase in |
| | | ~90% increase in root | root length over wild-type |
| | | length over wild-type | 54% to 77% higher |
| | | ~50% higher chlorophyll | chlorophyll content over |
| | | content over wild-type | wild-type |

Table 2 clearly demonstrates that the two genes of the instant disclosure showed positive results in different stress conditions like drought stress, salt stress and cold stress, thereby proving to be a better candidate for conferring multiple abiotic stress tolerance in plant.

### SEQUENCE LISTING

### <110> OSMANIA UNIVERSITY

<120> Polynucleotide, Polypeptide Sequences and Methods thereof

### <130> IP15067

<160> 4
<170> Patent In version 3.5
<210> 1
<211> 282

### <212> DNA

<213> Cajanus cajan

1. Polynucleotide Sequences as set forth in SEQ ID NO: 1 and SEQ ID NO: 2 or corresponding polypeptide sequences set forth in SEQ ID NO: 3 and SEQ ID NO: 4, respectively.

2. (canceled)

3. (canceled)

4. The sequences as claimed in claim 1, wherein the sequences are obtained from plant species Cajanus.

5. The sequences as claimed in claim 1, wherein the sequences impart abiotic stress tolerance in species selected from a group comprising Arabidopsis species and Tobacco species.

6. A vector comprising polynucleotide sequence as set forth in SEQ ID NO: 1 or SEQ ID NO: 2 or a combination thereof.

7. The vector as claimed in claim 6, wherein the vector is selected from a group comprising Agrobacterium based vector and E. coli based vector and comprises an antibiotic selection marker.

8. (canceled)

9. A recombinant cell comprising a vector as claimed in claim 6.

10. The recombinant cell as claimed in claim 9, wherein the cell is selected from a group comprising eukaryotic cells and prokaryotic cells.

11. A method of obtaining a recombinant cell, said method comprising acts of:
a) inserting polynucleotide sequence as set forth in SEQ ID NO: 1 or SEQ ID NO: 2 or a combination thereof in a vector; and
b) transforming a cell with the vector having said sequence to obtain the recombinant cell.

12. The method as claimed in claim 11, wherein the vector is selected from a group comprising Agrobacterium based vector and E. coli based vector; the cell is selected from a group comprising eukaryotic cells and prokaryotic cells; and the recombinant cell has abiotic stress tolerance.

13. A method of obtaining a transgenic plant comprising a polynucleotide sequence as set forth in SEQ ID NO: 1 or SEQ ID NO: 2 or a combination thereof, said method comprising acts of:
a) obtaining a recombinant cell by method as claimed in claim 11; and
b) inserting the recombinant cell into plant and culturing the plant to obtain the transgenic plant.
or comprising acts of:
a) transforming a plant with a vector comprising polynucleotide sequence as set forth in SEQ ID NO: 1 or SEQ ID NO: 2 or a combination thereof; and
b) culturing the transformed plant to obtain the transgenic plant.

14. The method as claimed in claim 13, wherein the transgenic plant has abiotic stress tolerance.

15. The method as claimed in claim 13, wherein the abiotic stress is selected from a group comprising high temperature, low temperature, drought, salinity, oxidative stress, osmotic stress, chemical agents and any combination thereof.

16. The method as claimed in claim 15, wherein the high temperature is ranging from about 40° C. to about 46° C. preferably about 42° C.; the low temperature is ranging from about 4° C. to about 12° C. preferably about 4° C.; the salinity is ranging from about 0.1M to about 1M preferably about 0.2M; the chemical agents are selected from a group comprising polyethylene glycol (PEG) and mannitol or a combination thereof.

17. The method as claimed in claim 16, wherein the polyethylene glycol (PEG) is at a concentration ranging from about 5% to about 25%, preferably about 20%, and the mannitol is ranging from about 100 mM to about 500 mM preferably about 300 mM.

18. A transgenic plant or plant part comprising polynucleotide sequences polypeptide sequences as claimed in claim 1 or combinations thereof.

19. The transgenic plant or plant part as claimed in claim 18, wherein the plant is selected from a group comprising Arabidopsis species and Tobacco species and wherein the plant art is selected from a group comprising plant cell, seed, shoot, root, leaf, flower and fruit.

20. (canceled)

21. The transgenic plant or plant part thereof as claimed in claim 18, wherein the transgenic plant or plant part posses abiotic stress tolerance.

22. The transgenic plant or plant part as claimed in claim 18, wherein the abiotic stress is selected from a group comprising high temperature, low temperature, drought, salinity, oxidative stress, osmotic stress, chemical agents and any combination thereof.

23. The transgenic plant or plant part as claimed in claim 22, wherein the high temperature is ranging from about 40° C. to about 46° C. preferably about 42° C.; the low temperature is ranging from about 4° C. to about 12° C. preferably about 4° C.; the salinity is ranging from about 0.1M to about 1M preferably about 0.2M; the chemical agents are selected from a group comprising polyethylene glycol (PEG) and mannitol or a combination thereof.

24. The transgenic plant or plant part as claimed in claim 23, wherein the polyethylene glycol (PEG) is at a concentration ranging from about 5% to about 25%, preferably about 20%, and the mannitol is ranging from about 100 mM to about 500 mM preferably about 300 mM.


SEQUENCE LISTING (4 sequences)

SEQ ID NO: 1 - DNA, 282 bp, Cajanus cajan
  gene (1)..(282)
atgtctgggatcatccacaagattgaggagaccttccatgggcacaagaaagaggagcacaaaggggagcatcacaaaggaggacacaaaggggaacaccatggtgaacacaaagggggacatcatggggagcacaaggagggttttatggacaaggtgaaggacaagatccatggtgatggagaacacaagggtgaggaaaagaagaaggagaagaagaagaagcacgagcatggacatgagcatcatggtcatgacagcagcagcagcgacagtgattaa

SEQ ID NO: 2 - DNA, 519 bp, Cajanus cajan
  gene (1)..(519)
atgcctaaccctaaggttttttttgacatggccatcgccggtcagcctgtcggccgtattgtatttgaactctacgccgacgtgactccccgcaccgccgagaacttccgtgctctctgcacaggcgagaagggagtcggccgcagcggccagcagctcaagtttaagggatcgtcttttcatcgcgtgatcccgaatttcatgtgtcaggggggagacgcagaggcccaagccaaggtgggcggtgagtcgttcttcgatatagaattcggggatgaatctttcgtgaagaagcacaccggtcctggtatcctctcgatggcgaacgatggcgaacgcgggtcctggaatcaattcttcatctgcaccgctaaaaccgattggcttgatggtaagcacgtggtcttcggtcaggtcgttggaggtatgaacgtggtaaaagacgttggaaaagtgggttcgagctccggaaggaaggagatcgagaaggttggatccgactgcggtcaaccctcttag

SEQ ID NO: 3 - protein, 93 aa, Cajanus cajan
  PEPTIDE (1)..(93)
MSGIIHKIEETFHGHKKEEHKGEHHKGGHKGEHHGEHKGGHHGEHKEGFMDKVKDKIHGDGEHKGEEKKKEKKKKHEHGHEHHGHDSSSSDSD

SEQ ID NO: 4 - protein, 172 aa, Cajanus cajan
  PEPTIDE (1)..(172)
MPNPKVFFDMAIAGQPVGRIVFELYADVTPRTAENFRALCTGEKGVGRSGQQLKFKGSSFHRVIPNFMCQGGDAEAQAKVGGESFFDIEFGDESFVKKHTGPGILSMANDGERGSWNQFFICTAKTDWLDGKHVVFGQVVGGMNVVKDVGKVGSSSGRKEIEKVGSDCGQPS